(19) United States
(12) Patent Application Publication
(10) Pub. No.: US 2026/0314745 A1
(43) Pub. Date: Oct. 15, 2026

(54) USER ADJUSTABLE ORTHOTIC APPARATUS AND METHOD

(71) Applicants: Daniel P. SJOGREN, Oxford, MA (US); Darrell A. POIRIER, Woodstock, CT (US)

(72) Inventors: Daniel P. SJOGREN, Oxford, MA (US); Darrell A. POIRIER, Woodstock, CT (US)

(21) Appl. No.: 19/173,890

(22) Filed: Apr. 9, 2025

(51) Int. Cl. A43B 7/142; A43B 7/143; A61F 5/14

(52) U.S. Cl. CPC A43B 7/142; A43B 7/143; A61F 5/14

(57) ABSTRACT

Present embodiments relate to a user adjustable orthotic system, apparatus or method for resolving bodily pain and more specifically pain related to muscular and/or skeletal problems including joint pain, back pain, knee pain, and so on caused from inadequate support of a person's muscular and/or skeletal systems, and/or mechanical imbalances throughout the spine, pelvis and/or feet. The embodiments include new apparatuses and methods for a wearer to manage their orthotics.

### TECHNICAL FIELD

Present embodiments relate to apparatuses and methods for resolving bodily pain and more specifically pain related to muscular and/or skeletal problems including joint pain, back pain, knee pain, and so on caused from inadequate support of a person's muscular and/or skeletal systems, and/or mechanical imbalances throughout the spine, pelvis and/or feet.

### BACKGROUND

As is well known, body pain related to musculoskeletal problems is common in many adults and children alike. Musculoskeletal pain can be caused by many things including injuries, fallen aches, poor muscle tone, etc. Doctors specialize in various areas of the body related to musculoskeletal pain including orthopedics, chiropractors, and podiatrists. These well-known and highly respected specialists provide care for patients with various musculoskeletal structural and mechanical problems in order to reduce and/or eliminate pain for many people.

Some of the solutions include braces, chiropractic adjustments, acupuncture, modalities and foot arch supports. Present embodiments focus on foot orthotics and arch support for correcting or easing musculoskeletal structural problems and/or pains.

Prior disclosures related to foot arch supports include several patents and publications that describe various insole devices and methods. For example, Lynden's U.S. Pat. Nos. 5,203,793, 5,101,580, and 4,674,206 detail insole devices with conformable material that mixes with water vapor in the atmosphere to become solid. Tansill's U.S. Pat. No. 4,128,951 describes a pliable arch support apparatus for footwear with a fill area and an inner container with a catalyst. Green's US Pub. 20060016103A1 introduces an outer and an inner container system where contents are combined and mixed by foot movement. Dieckhaus's U.S. Pat. No. 6,195,917 B1 discusses an arch support with foam layers, including a pliable top layer for comfort. Bressan's U.S. Pat. No. 3,398,469 describes a multi-layer apparatus with added intermediate foam for extra comfort and support. Dubner's U.S. Pat. No. 3,736,673 describes a moldable insole with a frangible envelope containing a two-phase solid foam synthetic resinous system and Leonard's US Pub. 20070048443 outlines a method for retrofitting shoes with cushioning from a curable composition. Davis et al. US Pub. 20150351488 teaches a fluid container for a sole layer of an article of footwear, wherein the fluid container includes a cap made of a pliable/flexible material such as thermoplastic polyurethane, polyamide, polyethylene, and polypropylene.

The previous disclosures, however, do not disclose or suggest the embodiments presented herein and do not solve the problems with regards to, for example, product shelf life and other advantages as disclosed in this specification.

### BRIEF SUMMARY

The orthotics with self-firming arch support disclosed herein comprises materials that, when initially installed into footwear or onto a summer footwear (e.g., slippers, sandals, and etc.), provides a system, apparatus or a method that first massages the foot arch area to stretch and loosen the foot arch system as a person walks, allowing the foot arch to move toward a natural position, then over time, the self-firming arch support changes characteristics and solidify to form a corrected arch support system. Moreover, the wearer has the ability to monitor and make further adjustments to the orthotics system.

Accordingly, an arch support apparatus for a footwear is described, comprising: a base layer and a top layer; a self-firming arch packet component configured to be in positioned between the base layer and the top layer wherein the self-firming arch packet component is configured to serve as an outer container and comprising: a fill material; a curing container containing a catalyst material, wherein the curing container is sealed by a glue cap; a flexible container with a frangible seal layer containing the curing container wherein the flexible container is chemically inactive to the curing container, the catalyst material, and the fill material; and wherein the arch support apparatus gets firmer when the self-firming arch packet component is squeezed, causing the glue cap to be released and pressurize the frangible container, further causing the frangible seal to open and release the catalyst material into the silicone fill material.

Furthermore, a method of providing an arch support apparatus for a footwear is also described, comprising: providing a base layer and a top layer; providing a self-firming arch packet component configured to be in positioned between the base layer and the top layer, wherein the self-firming arch packet component is configured to serve as an outer container and comprising: a fill material; a curing container containing a catalyst material, wherein the curing container is sealed by a glue cap; a flexible container with a frangible seal layer containing the curing container wherein the flexible container is chemically inactive to the curing container, the catalyst material, and the fill material; and wherein the arch support apparatus gets firmer when the self-firming arch packet component is squeezed, causing the glue cap to release and pressurize the frangible container, further causing the frangible seal to open and release the catalyst material into the silicone fill material.

### BRIEF DESCRIPTION OF THE DRAWINGS

FIG. 1 illustrates an arch support apparatus for a footwear.

FIG. 2 illustrates orthotic sensor data and control system.

FIG. 3 illustrates an inclined disc for orthotic area height adjustments.

FIG. 4 illustrates an arch area raise and lower apparatus.

FIG. 5 illustrates an orthotics paired with a smartphone.

FIG. 6 illustrates an orthotics smartphone software application (app).

FIG. 7 illustrates an orthotics adjustments using a smartphone software application.

FIG. 8 illustrates orthotic heating coils.

FIG. 9 illustrates wedges used for orthotic height adjustments.

FIG. 10 illustrates an arch shape heat sealing fixture machine.

FIG. 11 illustrates an example except of a Test Log File.

FIG. 12 illustrates layers of an adjustable orthotic apparatus/system.

### DETAILED DESCRIPTION

Described herein is a smart footwear orthotic including a self-firming arch support and foot support system offering a range of enhancements including data from sensors within the arch support apparatus that indicate pressure areas of a foot shoe combination helping users to understand what adjustments (e.g., using control data from an app) to use for maintaining comfort, proper posture, and avoiding potential injuries. Managing temperature and humidity within shoes can add to comfort and help with foot hygiene. Compared with prior disclosures, these advanced features offer significant benefits for athletes, individuals with medical conditions, and those who just seek enhanced comfort and performance in their footwear.

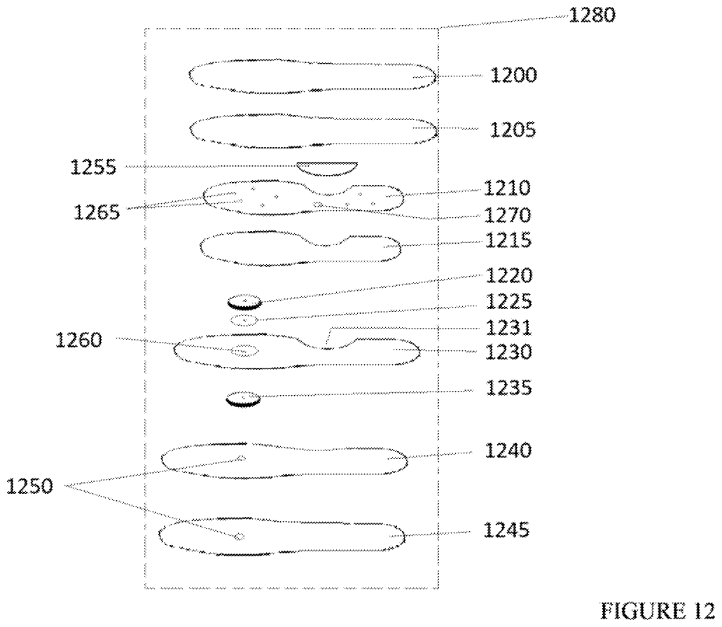

The user adjustable orthotic system 100 is made up of varying layers depending on the objectives of the wearer. Moreover, the system can include all layers or a subset of layers. FIG. 12 illustrates the layers included in a fully configured adjustable orthotic system (1280). The layered orthotic system (1280) in this example includes top (1200) and bottom (1245) layers, showing a cutout (1250) for a port for manual height adjustments. The layered orthotic system (1280) also include 3 layers of EVA type foam, layers (1205, 1230), showing with a cut out (1260) for a lifting component, and foam layer (1240) showing with a cutout (1250) for a port for manual adjustments) The layered orthotic system (1280) additionally include a lift plate (1215) of very thin (e.g., ~0.5 mm) flexible plastic, and an electronics layer (1210) that includes various sensors (1265), and microprocessor including a wireless function (1270) mounted on a very thin (e.g., ~0.5 mm) flexible plastic board. Additionally, the system (1280) includes various lifting components (1220, 1225, and 1235) described in more detail in the following paragraphs with the associated figures.

Also shown in FIG. 12 is a self-firming arch component (1255) that is installed between some layers and aside of other layers (e.g., EVA foam layers 1205, 1230 and 1240)). The self-firming arch system (1280) is accomplished by a mixture of certain components that separately are more fluid than solid but when mixed become solidified to a soft rubber consistency.

A user adjustable orthotic system as described herein does not require all of the above described layers shown in FIG. 12, if a user desires less functionality for example, the electronics layer may be omitted, and manual adjustments may be made. Or a self-firming arch component may be omitted using only the features of other areas of lift. Or as another example, all adjustment features could be eliminated leaving an orthotic system with a self-firming arch area.

The following paragraphs describe details of each component and layer of an exemplary system.

One component of the arch support system includes materials that, when initially installed into a footwear or onto a summer footwear (e.g., slippers, sandals and etc.), provides a system, an apparatus or a method that first massages the foot arch area to stretch and loosen the foot arch system as a person walks, allowing the foot arch to move toward a natural position, then over time, the arch support changes characteristics and solidify to form a corrected self-firming type of arch support system. Problems that can arise of such a system include unpleasant odors from the materials being used, a premature firming of the arch support area, and a reduce storage time and shelf life. Objectives of this disclosure are, among others, to provide solutions to solve these problems.

Prior disclosure (e.g., Tansill U.S. Pat. No. 4,128,951) describes a two container pliable arch support apparatus for footwear with an outer container with a fill area and a frangible inner container with a catalyst that when the two materials are mixed would result in firming. However, Tansill's method is incomplete with regards to odors, storage, shelf life, and a premature firming condition.

Other prior disclosures (e.g., Lynden's U.S. Pat. No. 5,203,793) use an aqueous (water) silicone emulsion material that when exposed to air (by removal of a barrier), causes the aqueous silicone to form to solid matter after a working time. However, the method is also incomplete with regards to odors, storage, shelf life, and a premature firming condition.

As discovered by the present inventors, by use and experimentation, premature firming is a condition where an arch support area would firm on the shelf before the intended use. In other words, a full firming of the arch area occurred prior to the outer container fill area and a catalyst being combined and mixed.

Our investigations indicated that the causes of premature firming include adverse reactions between various pliable containers made of materials like polyethylene, polypropylene, thin plastics, food grade plastics, and vegetable-based materials when combined with fill materials such as silicone, glycerin, alcohol, mineral spirits, and vinegars as some examples tested. Issues observed included containers dissolving, leaking, outgassing, and seal failures.

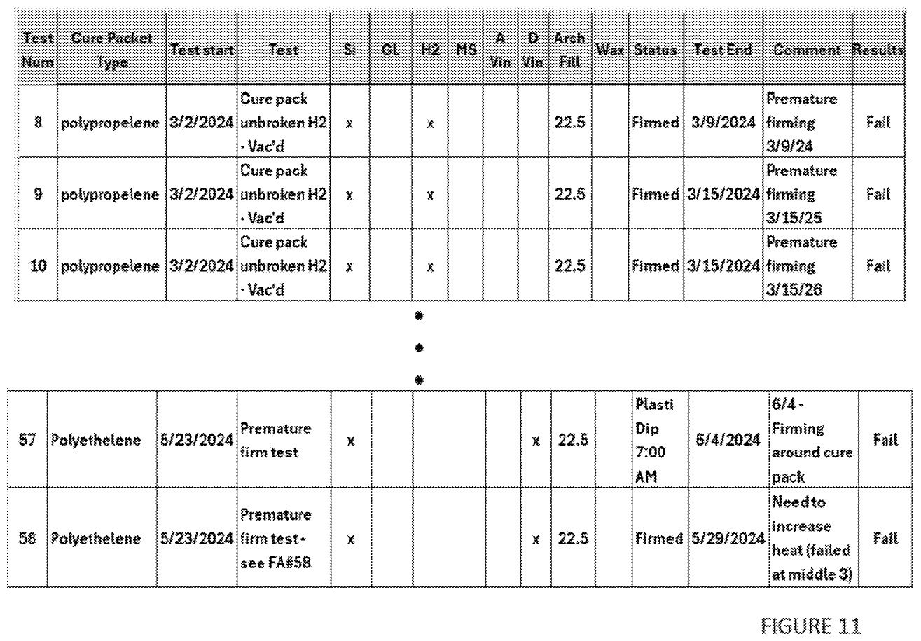

As a result, shelf life is typically about 14 days, with total failure (fully premature firmed arch area) typically occurring before 90 days by empirical testing (see FIG. 11, summary excerpt of test log).

Another failure observed by the present inventors was of the outer container contents (e.g., silicone) outgassing through pliable plastic containers causing a displeasing odor after a few days of storage with outgassing contributing to premature firming. Again, the prior disclosures did not address unpleasant odors nor this mode of premature firming conditions.

This disclosure adds enhancements to the prior disclosures to solve the problems of odors, premature firming, and limited shelf life.

The present inventors also determined that a solution was to incorporate a chemically inactive frangible third container to isolate the susceptible inner container materials from the outer container material (e.g., silicone). The frangible third container (e.g., a frangible pliable polypropylene container enclosing a polyethylene inner container containing a catalyst residing in a pliable outer container with a fill of silicone effectively isolates the polyethylene inner container from the silicone, thus avoiding a reaction between the polyethylene container and glue from the silicone.

An additional enhancement was to incorporate an outer layer on the outer container to prevent outgassing and odors. Tested outer layers included sealant sprays, wax dipping, and various plastic bags that have coatings on them like biaxially oriented polyethylene terephthalate (BoPET), also known by one brand name as mylar and a good candidate for use.

Moreover, mylar can be removed from plastic containers with various cured glues like contact cement. This can be used as an advantage in the construction of orthotics to ensure desired placement of components allowing a fully cured arch material to be released from glued surfaces and to slightly float and settle into a comfortable foot arch and shoe position within an arch pocket between conformable foam layers.

Accordingly, enhancements disclosed herein to prevent odors, premature firming, limited shelf life, and settled in comfort include creating an arch packet component in the shape of a foot arch and may comprise:
1) Adding a chemically inactive frangible third container.
2) Using silicone and a distilled vinegar catalyst as the firming components.
3) Use an outer layer of mylar on the outer container.
4) Add an arch area pocket for a cured arch packet to initially float after full settling.

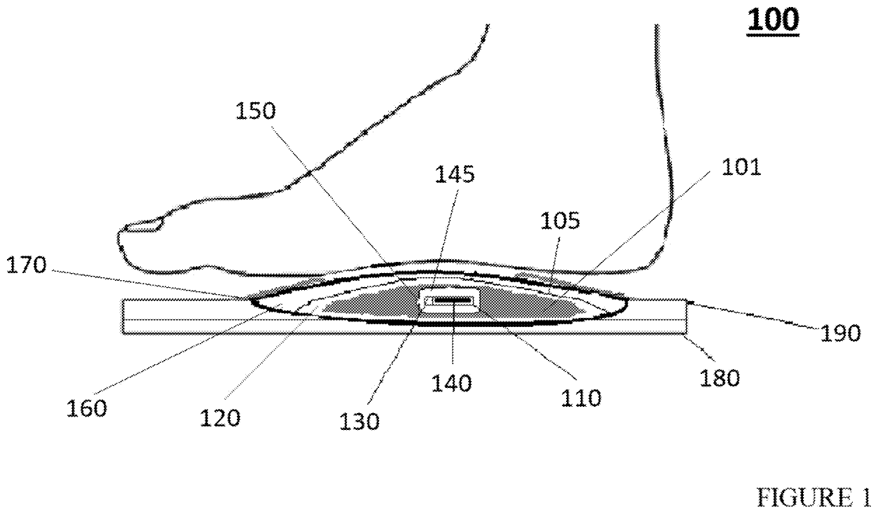

FIG. 1 illustrates a layered arch support orthotic apparatus or system (100) that includes a top layer (190), a base layer (180), and varying middle layers depending on the objective of the wearer. The layered orthotic apparatus or system (100) also contains a self-firming arch packet component (105). The self-firming arch packet component (105) is installed between the base layer (180) and the top layer (190). The self-firming arch packet component (105) acts as an outer container to contain two inner containers, a curing container (110) and a frangible container (145). The curing container (110) contains a catalyst material (140), wherein the curing container (110) is sealed by, for example, gluing a cap (130) (made of, e.g., plastic polyethylene) to seal one of its openings, or by filling one of its openings with glue cap (130). Examples of kinds of glue which may be used are e.g., Gorilla Heavy Duty Adhesive or Loctite® Shoe Glue.

The frangible container (145) contains the curing container (110). The frangible container (145) is flexible and is chemically inactive to the curing container (110), the catalyst material (140), and the fill material (101) within the self-firming arch packet component (105).

One example of a curing container (110) may be a pliable plastic polyethylene container (110) that contains distilled vinegar or a similar catalyst (140), and is sealed with a glue cap (130) as described above. One example of a frangible container (145) may be a polypropylene container (145), with a frangible seal layer (150). The frangible seal layer (150) is used as an inactive layer between the catalyst's polyethylene curing container (110) and the silicone fill material (101) within the self-firming arch packet component outer container (105). The polypropylene container (145) also provides additional protection from premature firming if the glue cap (130) should be compromised from excessive handling by containing the catalysts material (140) from penetrating into the outer container's (105) fill material (101).

The self-firming arch packet component (105) includes a layer of biaxially oriented polyethylene terephthalate (120) (commonly known as mylar) as the outer layer (120) of the container (105). The outer layer (120) may be glued in place or allowed to float within another arch support pocket (160). There is at least one foam layer (170) with a cutout area (FIG. 12 (1231)) for the self-firming arch packet component (105). The cutout area of the foam layer (FIG. 12 (1231)) creates a pocket area between the top layer (190) and the bottom layer (180) of the layered arch support orthotic apparatus (100). The self-firming arch packet component (105) resides in this pocket area and can be permanently glued in place using a contact cement, allowed to float within the pocket area, or be temporarily glued in place and then released to float within the pocket area over time using a temporary glue that releases over time, for example, using contact cement on a single side of the self-firming arch packet component (105), or alternatively using a strong glue that cause a separation of the mylar layer from the plastic layer of self-firming arch packet component (105) after the cure process has completed.

When the self-firming arch packet component (105) is squeezed, a chain reaction occurs causing the glue cap (130) to release and pressurize the frangible container (145), thus causing the frangible seal layer (150) to open and release the distilled vinegar (140) into the silicone fill material (101). This allows the distilled vinegar (140) to be mixed with the silicone fill material (101), and the mixed materials will solidify into a soft rubber consistency. The mixtures of silicon fill material (101) and distilled vinegar (140) are selected in an appropriate proportional in order for the mixed materials to solidify in approximately 2 hours, allowing the wearer time to use the orthotic in his or her shoes while the firming process occurs. In an exemplary embodiment, the proportion is 1 part distilled vinegar (140) to 25 part of silicone fill material (101).

One problem observed for creating a self-firming arch support component was a lack of tooling or products to seal a soft flexible plastic container that has the shape of a foot arch. Heat sealing equipment presently available on the market seals a straight edge with the equipment having a hot side and a pressure side, or in some cases may have two hot sides. Another method uses a hot wheel where plastic materials (sheets of plastic) run below the hot wheel, again providing seals only along a straight edge of the container. There are some hand-held wheel type heat seal products available, however these are non-exacting and insufficient for a manufacturing process with a very hot end that can be hazardous when in use.

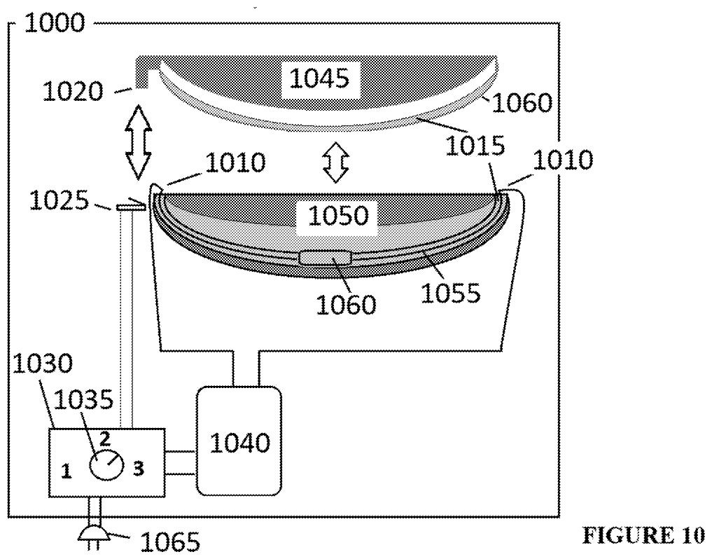

A solution was to create a foot arch shaped heat sealer (1000) as shown in FIG. 10. A heat seal fixture machine (1000) that can seal an arch packet open on multiple sides with fill materials installed, put between a hot side (a base in the shape of a foot arch with an arch pocket (1050) to hold inner components of a self-firming arch packet component) and the pressure side (1045) also in the shape of a foot arch. The base (1050) has a rounded outer edge in the shape of a foot arch with a heating element (1055) that sits on a heat resistant silicone (1015). Heat resistant silicone is also used on the pressure side (1015) of the fixture machine. Heat resistant cloth (1060) is applied over the heating element (partially shown) and on the top pressure side area (1060). Connected to the heating element are two voltage wires (1010) that are connected to a transformer (1040). The transformer and heating element are controlled using a controller (1030) with an adjustable timer (1035) that determines the length of time the arch heating element is energized. As the top pressure side (1045) is pressed upon the open arch packet top and bottom, a micro switch (1025) is switched on from a protrusion (1020) on the pressure side (1045) of the fixture creating a heat sealing of the self-firming arch packet component FIG. 1 (105).

Moving on to other layers of the orthotic system, another objective is to enable orthotic wearer's the ability to make personalized orthotic height adjustments with the benefit of sensed data from the adjustable orthotic system. Data collection includes foot contact pressure areas, foot stress distributions, temperature, and humidity sensors.

An exemplary system/apparatus may include two main components:
1) Orthotics that contain a microprocessor with memory, analog and digital IO pins, wireless communication, a plurality of pressure sensors configured to use a single IO pin and a ground (negative) connection, and the ability to make manual adjustments and/or adjustments using motors and software.
2) An orthotics smartphone application (app) that provides data including sensor data and user control data of the adjustable orthotic system.

To use a minimal amount of microprocessor IO pins, a single serial wire for multiple sensors is used. Sensors can include variable resistance or in another configuration a sensor can operate like a switch (open/closed) with added resistor across each sensor to create the sensor network. For example, if a switch is open the resistor value is read, and when the switch is closed, the resistance value will be near zero ohms. In either example, using resistor values that are significantly electrically separated (for example 470, 1 k, 100 k, and 150 k ohms) would provide identifiable circuit results when read as shown in the chart below:

| Sensor number | Resistor values (Ohms) across |
|---|---|
| pressed (closed) | each sensor |
| 1 | 470 |
| 2 | 1,000 |
| 3 | 100,000 |
| 4 | 150,000 |
| None pressed | 251,470 |

In this example, if a reading of the serial circuit indicates a value of 251,470 Ohms, it can be identified as no sensors being pressed upon. If the value read is 151,470 Ohms, then sensor #3 is being pressed, or if the reading is 101,470 Ohms, then sensor #4 is pressed and so on.

This example illustrates single sensor press condition whereas a longer chart is generated to identify all possible sensor combinations. This type of serial configuration can be applied to a serially wired grid system to provide greater granularity of foot data. The sensors may be designed to incorporate the various resistor values needed.

Other electronic component examples include a small form factor microprocessor (e.g., ATmega328) with a wireless function added (e.g., Bluetooth HC05/6), or an ESP32 with low energy bluetooth, small form factor batteries (e.g., CR2032's) that can be replaced, or a thin form factor lithium ion battery and a coil and charge circuit for wireless charging. Obviously, any number of different types of microprocessors or controllers could be used as people skilled in the art would recognize.

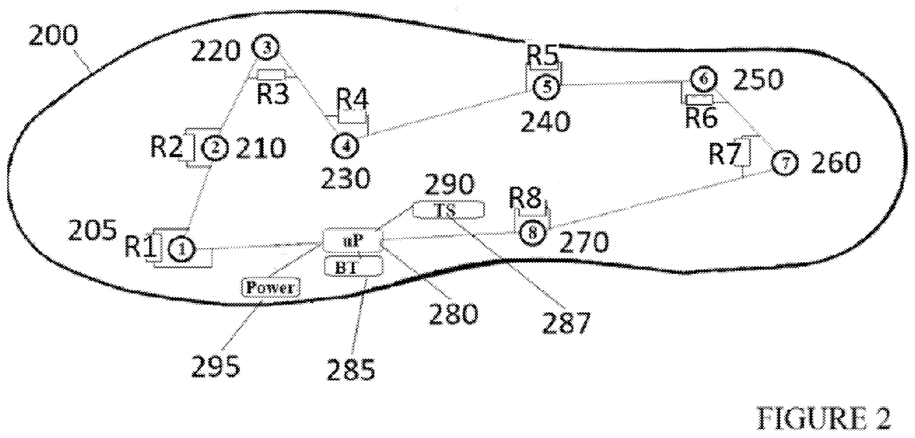

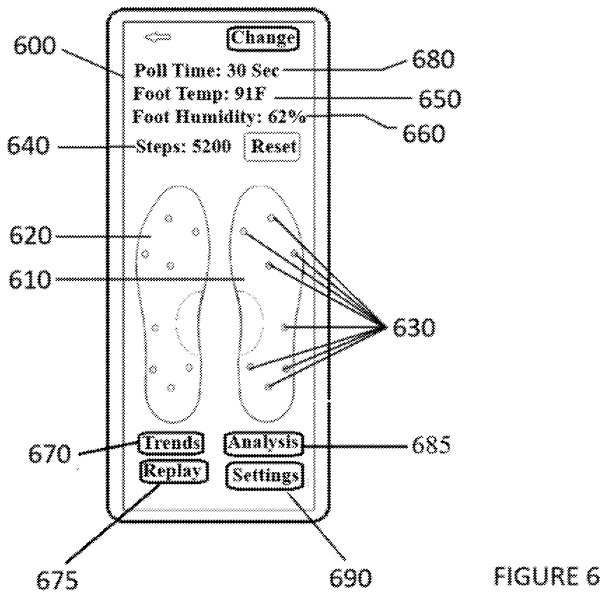

FIG. 2 illustrates an orthotic layer sensor network of switch/resistor pairs (200) including sensors 1-R1 (205), 2-R2 (210), 3-R3 (220), 4-R4 (230), 5-R5 (240), 6-R6 (250), 7 (260), and 8-R8 (270) placed at various positions and serially wired to a microprocessor (280) IO pin. Sensor status is read using microprocessor analog I/O pin (280) powered by a battery (295) where data and control are managed wirelessly (285) via an orthotics smartphone app FIG. 6 (600) communicating with software running on the microprocessor (280).

Bluetooth in each orthotic “bluetooth pairs” with a smartphone and interfaces with an orthotics smartphone app (FIG. 6, 600) that provides monitoring and sensing data from each foot sensor network, temperature sensor, and humidity sensor providing orthotic data for each foot to the wearer.

The smartphone app (600) provides other features like number of steps, graphing of data trends over time, and could be used by a doctor for diagnostic purposes or wearer behavior training similar to products that come from companies that have focused on data collection like; Feetme, Foot Logger, Boltt Sports Technologies and Altra Footwear.

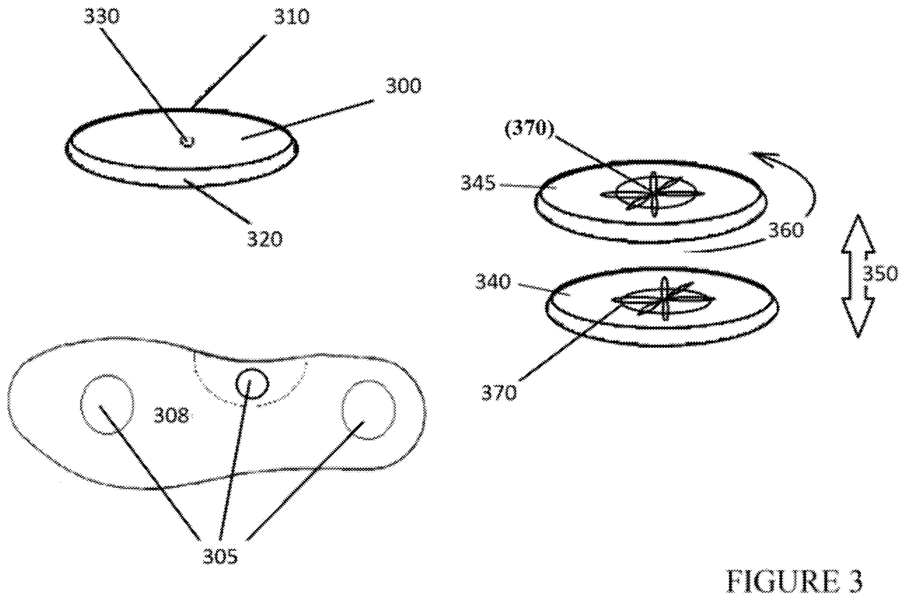

One objective of the present embodiments is to enhance what's presently available by adding an adjustable arch support system focused on user controllable orthotics. Methods for adjusting orthotic areas can include manual control, electronic control, or a combination of both. FIG. 3 illustrates height adjustments discs (300) which vary in thickness around the diameter (320) and located (305) as a layer (308) of an orthotic assembly FIG. 12, (1230) that can be placed in various locations depending on the objective of need (305). Additional discs can be stacked for increased height.

FIG. 3 illustrates an adjustment component such as a disc (300) that has a thinner edge (310) and thicker edge (320) (e.g., tapered circular wedge). Two discs are configured face to face, one being fixed (340) and another that is non-fixed (345) in this example allowing orthotic height adjustments (350) to be made as disc is rotated (360). The disc may be turned manually with a small tool like a screwdriver through an access port on the bottom of the orthotic using a center slot (330), while another disc is in a fixed position and cannot be rotated (340). Alternatively, the disc may be rotated electronically by applying voltage from a microprocessor FIG. 2 (280) to a coil winding within each disc (370) or alternatively a magnet in one of the disc. Thin washers (FIG. 12, 1225) may be added between disc and/or layers to better isolate moving parts providing less friction.

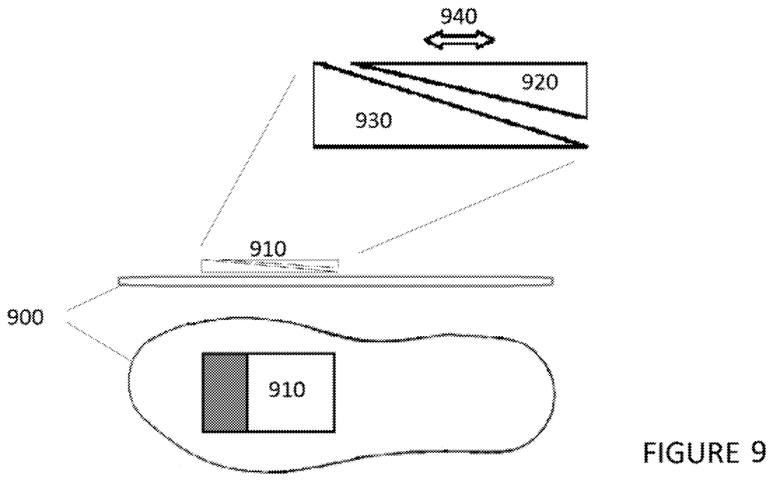

As an alternative to discs, wedges can be used to raise or lower an orthotic area. FIG. 9 illustrates an orthotic (900) with a wedge assembly (multiple wedges) located near the middle of the orthotic to be raised or lowered (910). Using a manual method or micro motors method as previously described, one side of the wedge would be in a fixed position (930) and another wedge (920) facing the fixed position wedge (930) would be moveable (940) causing the lifting or lowering action.

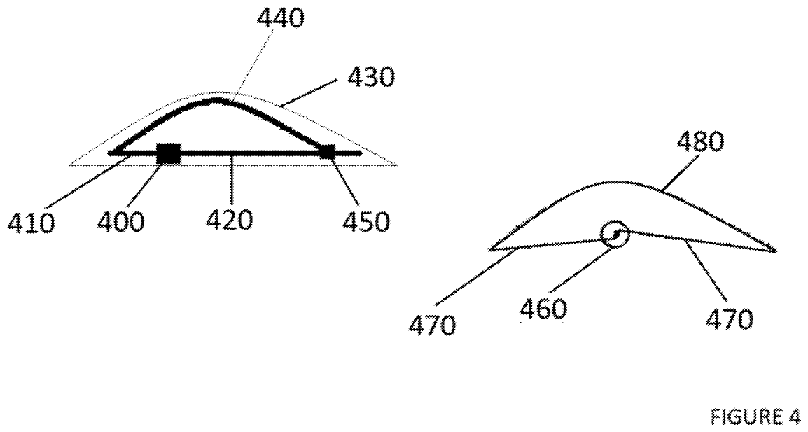

Referring to FIG. 4, another method for lifting an orthotic area would be to use a micro motor (400) that spins an attached threaded rod (420) using the microprocessor FIG. 2 (280). In this case the threaded rod (420) is used to raise an arch area by pulling or pushing a flexible metal band that has material affixed to it (430) like rubber, silicone, leather, cloth, etc. The steel band (440) affixed to the flexible material (430) is fixed at one end to the threaded rod with a threaded nut (450) causing movement when the threaded rod is rotated and fixed at the opposite end to a stationary position (410). A raise or lower adjustment may also be done using a tool to manually turn the threaded rod (420) through an access port on the orthotic. In yet another method, a micro motor (460) with a geared down shaft coils a small cable (470) that pulls tight opposite ends of an internal steel band lifting or lowering a material like rubber, silicone, leather, cloth, etc. (not shown).

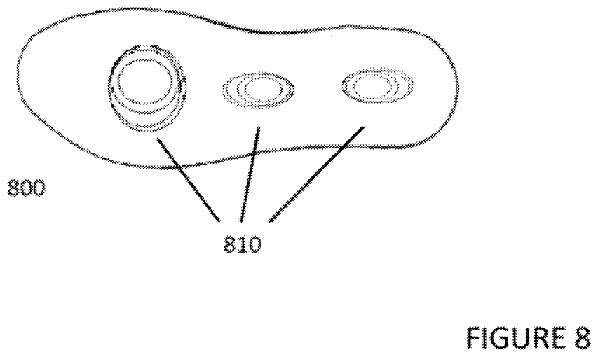

Other comfort functionality could be added, like microprocessor FIG. 2 (280) controlled heating wires FIG. 8 (810) that operate with a feedback loop from the temperature sensor FIG. 2 (290) to keep feet at a desired specific temperature. The temperature is set using the orthotics smartphone app FIG. 6 (600). A relay or transistor activated by microprocessor control are used to drive the current for the heating coil.

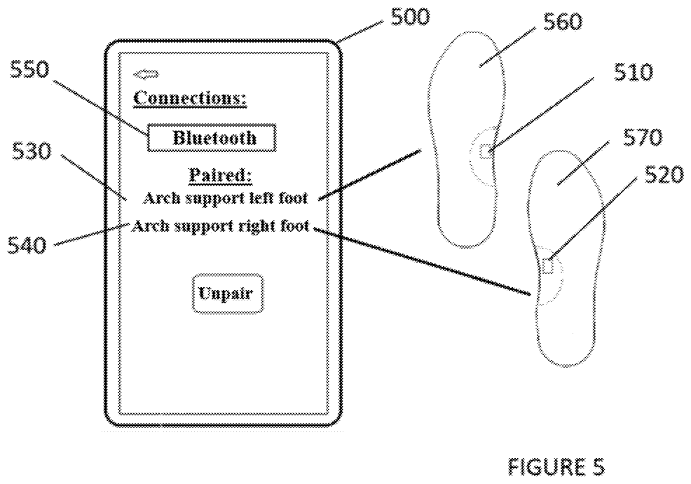

As previously stated, a smartphone is used to wirelessly connect to each orthotic for monitor and control purposes. Referring to FIG. 5, each orthotic (560) and (570) has a Bluetooth transceiver (510) and (520) that connects to a smartphone (500) with bluetooth components and functionality (550), allowing each orthotic a wireless paired connection:
a. Paired with left foot (530)
b. Paired with right foot (540)

Once the phone is paired with each orthotic, the orthotics smartphone app can receive and send data to each orthotic. Referring to FIG. 6, the orthotics smartphone app (600) displays a graphic of the left (620) and right (610) orthotic including a layout with indicators for each sensor (630) that are colored, for example, green with no foot pressure or red when foot pressure is applied.

The orthotics smartphone app provides features such as:
1. Images including each pressure switch or combination thereof in real time (630).
2. Logging of foot statistics:
   a. Steps (640)
   b. Replay of steps on sensors (675)
   c. Temperature (650)
   d. Pressure point areas (630)
   e. Humidity (660)
3. Histogram trends (670):
   Provides foot activity and readings over time. The time is adjustable (680), allowing readings to be taken at any desired intervals. Graphs are provided to indicate foot statistics over time.
4. AI training data and analysis of trends (685):
   Large collections of data from groups of people could be used for the training of artificial intelligence providing potentially valuable data to doctors, shoe manufacturers, and shoe wearers.
5. Adjustable support features using the “Settings” (690) button on the orthotics smartphone app.

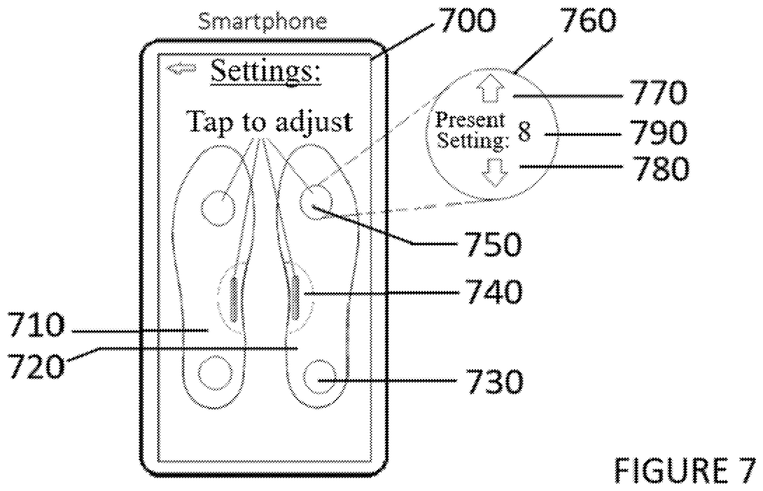

As previously mentioned, the orthotics may contain mechanisms that allow adjustments to be made for heights for arch and other areas of the foot. These adjustments can be made manually through an access port on the bottom of the orthotic (FIG. 12 (1250) or alternatively can be done using the orthotics smartphone app as shown in FIG. 7.

Referring to FIG. 7, the smartphone app (700) displays a graphic of the left foot (710) and the right foot (720). In this example there are 3 adjustment areas for each orthotic including the heel area (730), the arch area (740), and the ball of the foot area (750). If a user taps one of these areas on the display an adjustment settings option is displayed (760) where adjustments to raise (770) or lower (780) an area of the orthotic can be made, and also an indication of the present setting (790) is displayed based on a ring of contacts on the disc, coil rotary position, or a read from memory saved of the last position set.

Reference to “one embodiment” or “an embodiment” or “one implementation” or “an implementation”, as well as other variations thereof, means that a particular feature, structure, characteristic, and so forth described in connection with the embodiment is included in at least one embodiment. Thus, the appearances of the phrase “in one embodiment” or “in an embodiment” or “in one implementation” or “in an implementation”, as well any other variations, appearing in various places throughout this application are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as is clear to one of ordinary skill in this and related arts, for as many items as are listed.

We describe a number of embodiments. Features of these embodiments can be provided alone or in any combination.

1. An arch support apparatus for a footwear, comprising:
a base layer and a top layer;
a self-firming arch packet component configured to be positioned between the base layer and the top layer wherein the self-firming arch packet component is configured to serve as an outer container and the outer container comprising:
a fill material;
a curing container containing a catalyst material, wherein the curing container is sealed by a glue cap;
a flexible container with a frangible seal layer containing the curing container wherein the flexible container is chemically inactive to isolate the catalyst material from the fill material; and
wherein the arch support apparatus is configured to get gets firmer when the self-firming arch packet component is squeezed, causing the glue cap to be released and pressurize the frangible container, further causing the frangible seal to open and release the catalyst material into silicone fill material, wherein the catalyst material is distilled vinegar.

2. The arch support apparatus of claim 1, further comprising at least one foam layer with a cutout area for the self-firming arch packet component.

3. The arch support apparatus of claim 1, further comprising an outer layer of biaxially oriented polyethylene terephthalate for the self-firming arch packet component.

4. The arch support apparatus of claim 1, further comprising a pocket wherein the cured self-firming arch packet component is configured to float within the pocket.

5. The arch support apparatus of claim 1, further comprising:
at least one sensor; and
a microprocessor connected to the at least one sensor.

6. The arch support apparatus of claim 5 wherein data from the arch support apparatus are transmitted to/from the at least one sensor via the microprocessor using a wireless connection.

7. The arch support apparatus of claim 6 wherein the data are transmitted to/from a smartphone software application via the wireless connection.

8. (canceled)

9. A method of providing an arch support apparatus for a footwear, comprising:
providing a base layer and a top layer;
providing a self-firming arch packet component configured to be positioned between the base layer and the top layer, wherein the self-firming arch packet component is configured to serve as an outer container and outer container comprising:
a fill material;
a curing container containing a catalyst material, wherein the curing container is sealed by a glue cap;
a flexible container with a frangible seal layer containing the curing container wherein the flexible container is chemically inactive to isolate the catalyst material from the fill material; and
wherein the arch support apparatus is configured to get gets firmer when the self-firming arch packet component is squeezed, causing the glue cap to release and pressurize the frangible container, further causing the frangible seal to open and release the catalyst material into silicone fill material, wherein the catalyst material is distilled vinegar.

10. The method of claim 9 wherein the arch support apparatus further comprising at least one foam layer with a cutout area for the self-firming arch packet component.

11. The method of claim 9 wherein the arch support apparatus further comprising an outer layer of biaxially oriented polyethylene terephthalate for the self-firming arch packet component.

12. The method of claim 9 wherein the arch support apparatus further comprising a pocket wherein the cured self-firming arch packet component is configured to float within the pocket.

13. The method of claim 9 further comprising:
providing at least one sensor and a microprocessor connected to at least one sensor for the arch support apparatus.

14. The method of claim 13 wherein data from the arch support apparatus are transmitted to/from sensor via the microprocessor using a wireless connection.

15. The method of claim 14 wherein the data are transmitted to/from a smartphone software application via the wireless connection.

16. (canceled)

17. An apparatus for creating a self-firming arch component comprising:
a foot arch shape base unit comprising:
a heating element;
a heat resistant material covering the heating element;
an electrical transformer providing voltage to the heating element;
an electrical controller;
a micro switch;
a foot arch shape pressure unit comprising:
a protrusion; and
a heat resistant material;
wherein pressure is applied to top and bottom layers of an arch support packet and the top and bottom layers are heat sealed when the protrusion makes contact with the micro switch.

18. The arch support apparatus of claim 1, further comprising:
a sensor network; and
a microprocessor connected to the sensor network.

19. The arch support apparatus of claim 1 wherein the sensor network comprising serially connected sensors and the sensor network is also serially connected to the microprocessor.

20. The method of claim 9 further comprising:
providing at least one sensor network and a microprocessor serially connected to the at least one sensor network for the arch support apparatus.

21. (canceled)

22. (canceled)
